(19) United States
(12) Patent Application Publication
(10) Pub. No.: US 2021/0219847 A1
(43) Pub. Date: Jul. 22, 2021

(54) METHOD AND SYSTEM FOR PURPLE LIGHT IMAGING

(71) Applicant: MOBILEODT, LTD., Tel Aviv (IL)

(72) Inventors: Amir Shlomo BERNAT, Moshav Mazor (IL); David LEVITZ, Tel Aviv (IL); Frank John BOLTON, Petah Tikva (IL)

(21) Appl. No.: 17/055,594

(22) PCT Filed: May 14, 2019
(86) PCT No.: PCT/IL2019/050548

§ 371 Date: Nov. 15, 2020

Related U.S. Application Data
(60) Provisional application No. 62/670,938, filed on May 14, 2018.

(51) Int. Cl. A61B 5/0205; A61B 5/026; A61B 5/1455; A61B 5/00; A61B 5/145

(52) U.S. Cl. CPC A61B 5/0205; A61B 5/0261; A61B 5/14546; A61B 5/489; A61B 5/4875; A61B 5/14552

(57) ABSTRACT

A method for detecting tissue abnormality in a tissue sample, comprising: illuminating a tissue sample in vivo with a first light beam having a wavelength selected from the range 390-430 nanometer (nm); applying contrast agent to the tissue sample; capturing a one or more images of the tissue sample; and detecting tissue abnormality based on at least one of color changes in the tissue sample and blood vessel features in the tissue sample appearing in said one or more images.

### CROSS-REFERENCE TO RELATED APPLICATIONS

This application claims the benefit of priority to U.S. Provisional Patent Application No. 62/670,938, filed May 14, 2018, entitled “Method and System for Purple Light Imaging”, the contents of which are incorporated herein by reference.

### FIELD OF THE INVENTION

The invention relates generally to medical imaging systems.

### BACKGROUND

Many medical diagnostic procedures rely on visual examination for detecting abnormalities or disease, through imaging of an area under observation. For example, colposcopy, or visual examination of the cervix, can discern areas where there is a suspicion of pathology in the cervical tissue. However, direct visual observation alone is often inadequate for identification of abnormalities in a tissue. In some instances, a contrast agent, such as acetic acid, is applied to enhance the differences in appearance between normal and pathological areas. Areas with a high risk of neoplasia, or cancer, will appear as varying degrees of whiteness, because aceto-whitening correlates with higher nuclear density. In addition, another important feature considered in colposcopic diagnosis is vascular patterns, such as punctuation and mosaicism present in the cervix region. Such patterns are a marker of abnormal architecture of blood vessels, and their presence is significantly correlated with the existence of pre- and cancerous lesions of the cervix.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in accordance with an embodiment, A method for detecting tissue abnormality in a tissue sample, the method comprising: illuminating a tissue sample in vivo with a first light beam from a light beam source, said light beam having a wavelength selected from the range of 390-430 nanometers (nm); applying a contrast agent to the tissue sample; capturing one or more images of the tissue sample; and detecting tissue abnormality based on at least one of: color changes in the tissue sample, and blood vessel features in the tissue sample, appearing in said one or more images.

In some embodiments, the capturing comprises using an imaging device configured for detecting at least one of RGB (red-green-blue), monochrome, ultraviolet (UV), near infrared (NIR), and short-wave infrared (SWIR) spectral data.

In some embodiments, the imaging device comprises a digital imaging sensor selected from the group consisting of: complementary metal-oxide-semiconductor (CMOS), charge-coupled device (CCD), Indium gallium arsenide (InGaAs), and polarization-sensitive sensor element.

In some embodiments, the imaging device is configured for capturing said one or more images along a direct optical path from said tissue sample. In some embodiments, the imaging device is coupled to a fiber optic light guide for directing at least one of reflectance and fluorescence from the tissue sample to the imaging device.

In some embodiments, the illuminating comprises illuminating said tissue sample directly by said light beam source. In some embodiments, the illuminating comprises using a fiber optic light guide for transmitting light from the light beam source to said tissue sample.

In some embodiments, at least one of said one or more images are being captured before the step of applying said contrast agent, at least one of said one or more images are being captured after the step of applying said contrast agent, and wherein said detection is based at least in part on a comparison between said images captured before and after the step of applying said contrast agent.

In some embodiments, at least some of said one or more images are being captured at a specified time after the step of applying said contrast agent, wherein said specified time is between 1 and 600 seconds after the step of applying said contrast agent.

In some embodiments, the capturing comprises capturing a one or more RGB images and changing one or more amplification ratios between the RGB channels, wherein said detecting is further based on detecting fluorescence emitted by said tissue sample appearing in at least some of said one or more images. In some embodiments, the fluorescence is in at least one wavelength selected from the ranges 490-580 nm and 600-750 nm.

In some embodiments, the step of illuminating comprises illuminating said tissue sample with said first light beam and with a second light beam having a different wavelength to said first light beam.

In some embodiments, said first light beam and said second light beam illuminate the tissue sample simultaneously. In some embodiments, said first light beam and said second light beam illuminate the tissue sample sequentially, wherein at least some of said one or more images are being captured during a period for which said first light beam is illuminating the tissue sample, and at least some of said one or more images are being captured during a period for which said second light beam is illuminating the tissue sample.

In some embodiments, the capturing further comprises using two or more imaging devices, wherein each of said imaging devices is configured for acquiring image data in different one or more spectral bands.

In some embodiments, the method further comprises using at least one of a dichroic mirror and a beam splitter. In some embodiments, the at least one dichroic mirror has a cutoff wavelength selected from the group consisting of 430 nm, 580-660 nm, and 800 nm. In some embodiments, the method further comprises using confocal imaging.

In some embodiments, the second light beam has a wavelength selected from the range 495-570 nm, wherein said detecting is further based on detecting blood vessel features below a superficial layer of said tissue sample appearing in at least some of said one or more images.

In some embodiments, the step of illuminating further comprises illuminating said tissue sample with a third light beam, wherein each of said second and third light beams has a wavelength selected from the range 585-720 nm, and wherein said detecting is further based on determining a value of oxygen saturation of the blood in said tissue sample.

In some embodiments, the second light beam has a wavelength selected from the range 900-3000 nm, wherein said detecting is further based on determining a value of fluid accumulation in said tissue sample.

In some embodiments, the second light beam has a wavelength selected from the range 100-390 nm, wherein said detecting is further based on measuring fluorescence emitted by one or more excited fluorophores in said tissue sample.

In some embodiments, the second light beam comprises a projection system configured illuminating said tissue sample with spatially-structured light, wherein said detecting is further based, at least in part, on depth-resolved measuring of one or more of tissue blood concentration, tissue blood oxygenation, tissue water fraction, tissue perfusion, collagen, lipids, and exogenous agents. In some embodiments, the spatially-structured light is configured for performing spatial frequency domain imaging (SFDI). In some embodiments, the depth-resolved measuring comprises adjusting at least one of light frequency, wavelength selection, and amplitude modulation of said spatially-structured light.

In some embodiments, the method further comprises illuminating said tissue sample with a first polarized light source for producing light beams with at least a first polarization feature, and a second polarized light sources for producing light beams with at least a second polarization feature; wherein said capturing comprises capturing, by a polarization sensitive sensor element (SE), a plurality of images of the tissue sample, wherein at least some of said plurality of images are being captured during a period for which said first polarized light source is illuminating the tissue sample, and at least some of said plurality of images are being captured during a period for which said second polarized light source is illuminating the tissue sample.

In some embodiments, the first and second polarized light sources are configured for illuminating the tissue sample alternately based on one or more predetermined time intervals. In some embodiments, the capturing further comprises processing said plurality of images to separate light from a superficial single-scattering layer of the tissue sample and light from a deeper diffuse layer of the tissue sample.

In some embodiments, the method comprises using a non-total internal reflection (TIR) birefringent polarizing prism (BPP) optically coupled to the tissue sample and configured for guiding the light waves returning from the tissue sample.

In some embodiments, the method further comprises using one or more optical retarders along the optical path between the first and second polarized light sources and the tissue sample, wherein said one or more optical retarders are configured as a Mueller matrix imaging polarimeter (MMIP) for determining a partial or complete polarization matrix of the image.

There is also provided, in accordance with some embodiments, a system comprising a light beam source; an imaging device; at least one hardware processor; and a non-transitory computer-readable storage medium having stored thereon program instructions, the program instructions executable by the at least one hardware processor to: operate said light beam source to illuminate a tissue sample with a first light beam having a wavelength selected from the range 390-430 nanometer (nm), wherein said tissue sample is applied with a contrast agent, and operate said imaging device to capture one or more images of the tissue sample; wherein tissue abnormality is detectable based on at least one of color changes in the tissue sample and blood vessel features in the tissue sample appearing in said one or more images.

In some embodiments, the imaging device is configured for detecting at least one of RGB (red-green-blue), monochrome, ultraviolet (UV), near infrared (NIR), and short-wave infrared (SWIR) spectral data.

In some embodiments, the imaging device comprises a digital imaging sensor selected from the group consisting of: complementary metal-oxide-semiconductor (CMOS), charge-coupled device (CCD), Indium gallium arsenide (InGaAs), and polarization-sensitive sensor element.

In some embodiments, the imaging device is configured for capturing said one or more images along a direct optical path from said tissue sample.

In some embodiments, the system further comprises alight guide configured for directing at least one of reflectance and fluorescence from the tissue sample to the imaging device.

In some embodiments, the light beam source is configured for illuminating said tissue sample directly.

In some embodiments, the further comprises a light guide configured for transmitting light from said light beam source to said tissue sample.

In some embodiments, the instructions comprise operating said imaging device to capture at least some of said one or more images before said tissue sample is applied with said contrast agent, wherein said detecting is based at least in part on a comparison between said images captured before the step of applying said contrast agent and said images captured after the step of applying said contrast agent.

In some embodiments, the instructions comprise operating said imaging device to capture at least some of said one or more images at a specified time period after said tissue sample is applied with said contrast agent, wherein said specified time period is between 15 and 600 seconds.

In some embodiments, at least some of said one or more images are RGB images, wherein said instructions comprise changing one or more amplification ratios between RGB channels of said RGB images, wherein said detecting is further based on detecting fluorescence emitted by said tissue sample appearing in at least some of said one or more images. In some embodiments, the fluorescence is in at least one wavelength selected from the ranges 490-580 nm and 600-750 nm.

In some embodiments, the system further comprises a second light source configured for illuminating said tissue sample with a second light beam, wherein said second light beam has a different wavelength to said first light beam.

In some embodiments, the instructions further comprise operating said first and second light sources for illuminating said tissue sample simultaneously with said first light beam and said second light beam.

In some embodiments, the instructions further comprise operating said first and second light sources for illuminating said tissue sample sequentially with said first light beam and said second light beam, wherein at least some of said one or more images are being captured during a period for which said first light beam is illuminating the tissue sample, and at least some of said one or more images are being captured during a period for which said second light beam is illuminating the tissue sample.

In some embodiments, the system comprises two or more imaging devices, wherein each of said imaging devices is configured for acquiring image data in different one or more spectral bands.

In some embodiments, the system further comprising at least one of a dichroic mirror and a beam splitter. In some embodiments, the at least one dichroic mirror has a cutoff wavelength selected from the group consisting of 430 nm, 580-660 nm, and 800 nm. In some embodiments, the system further comprises confocal imaging means.

In some embodiments, the second light beam has a wavelength selected from the range 495-570 nm, wherein said detecting is further based on detecting blood vessel features below a superficial layer of said tissue sample appearing in at least some of said one or more images.

In some embodiments, the system further comprises a third light source configured for illuminating said tissue sample with a third light beam, wherein each of said second and third light beams has a wavelength selected from the range 585-720 nm, and wherein said detecting is further based on determining a value of oxygen saturation of the blood in said tissue sample.

In some embodiments, the second light beam has a wavelength selected from the range 900-3000 nm, wherein said detecting is further based on determining a value of fluid accumulation in said tissue sample.

In some embodiments, the second light beam has a wavelength selected from the range 100-390 nm, wherein said detecting is further based on measuring fluorescence emitted by one or more excited fluorophores in said tissue sample.

In some embodiments, the system further comprises a projection system configured for illuminating said tissue sample with spatially-structured light, wherein said detecting is further based, at least in part, on depth-resolved measuring of one or more of tissue blood concentration, tissue blood oxygenation, tissue water fraction, tissue perfusion, collagen, lipids, and exogenous agents.

In some embodiments, the spatially-structured light is configured for performing spatial frequency domain imaging (SFDI). In some embodiments, the depth-resolved measuring comprises adjusting at least one of light frequency, wavelength selection, and amplitude modulation of said spatially-structured light.

In some embodiments, the system further comprises a first polarized light source for producing light beams with at least a first polarization feature, and a second polarized light source for producing light beams with at least a second polarization feature, wherein said imaging device comprises a polarization sensitive sensor element (SE), wherein at least some of said plurality of images are being captured during a period for which said first polarized light source is illuminating the tissue sample, and at least some of said plurality of images are being captured during a period for which said second polarized light source is illuminating the tissue sample.

In some embodiments, the first and second polarized light sources are configured for illuminating the tissue sample alternately based on one or more predetermined time intervals. In some embodiments, the instructions further comprise processing said one or more images to separate light from a superficial single-scattering layer of the tissue sample and light from a deeper diffuse layer of the tissue sample. In some embodiments, the system further comprises a non-total internal reflection (TIR) birefringent polarizing prism (BPP) optically coupled to the tissue sample and configured for guiding the light waves returning from the tissue sample. In some embodiments, the system further comprises one or more optical retarders along the optical path between the first and second polarized light sources and the tissue sample, wherein said one or more optical retarders are configured as a Mueller matrix imaging polarimeter (MMIP) for determining a partial or complete polarization matrix of the image.

There is further provided, in accordance with some embodiments, a computer program product, the computer program product comprising a non-transitory computer-readable storage medium having program code embodied therewith, the program code executable by at least one hardware processor to operate a light beam source to illuminate a tissue sample in vivo with a first light beam having a wavelength selected from the range 390-430 nanometer (nm), wherein said tissue sample is applied with a contrast agent; and operate an imaging device to capture one or more images of the tissue sample; wherein tissue abnormality is detectable based on at least one of color changes in the tissue sample and blood vessel features in the tissue sample appearing in said one or more images.

In some embodiments, the imaging device is configured for detecting at least one of RGB (red-green-blue), monochrome, ultraviolet (UV), near infrared (NIR), and short-wave infrared (SWIR) spectral data.

In some embodiments, the imaging device comprises a digital imaging sensor selected from the group consisting of: complementary metal-oxide-semiconductor (CMOS), charge-coupled device (CCD), Indium gallium arsenide (InGaAs), and polarization-sensitive sensor element.

In some embodiments, the imaging device is configured for capturing said one or more images along a direct optical path from said tissue sample. In some embodiments, the imaging device is coupled to a light guide for directing at least one of reflectance and fluorescence from the tissue sample to the imaging device.

In some embodiments, the light beam source is configured for illuminating said tissue sample directly. In some embodiments, the light beam source is coupled to a light guide configured for transmitting light from said light beam source to said tissue sample.

In some embodiments, the program code comprises operating said imaging device to capture at least some of said one or more images before said tissue sample is applied with said contrast agent, wherein said detecting is based at least in part on a comparison between said images captured before the step of applying said contrast agent and said images captured after the step of applying said contrast agent.

In some embodiments, the program code comprises operating said imaging device to capture at least some of said one or more images at a specified time period after said tissue sample is applied with said contrast agent, wherein said specified time period is between 1 and 600 seconds.

In some embodiments, at least some of said one or more images are RGB images, wherein said program code comprises changing one or more amplification ratios between RGB channels of said RGB images, and wherein said detecting is further based on detecting fluorescence emitted by said tissue sample appearing in at least some of said one or more images. In some embodiments, the fluorescence is in at least one wavelength selected from the ranges 490-580 nm and 600-750 nm.

In some embodiments, the program code further comprises operating a second light beam source for illuminating said tissue sample with a second light beam, wherein said second light beam has a different wavelength to said first light beam.

In some embodiments, the program code further comprises operating said first and second light sources for illuminating said tissue sample simultaneously with said first light beam and said second light beam.

In some embodiments, the program code further comprises operating said first and second light sources for illuminating said tissue sample sequentially with said first light beam and said second light beam, wherein at least some of said one or more images are being captured during a period for which said first light beam is illuminating the tissue sample, and at least some of said one or more images are being captured during a period for which said second light beam is illuminating the tissue sample.

In some embodiments, the program code further comprises operating two or more imaging devices, wherein each of said imaging devices is configured for acquiring image data in different one or more spectral bands.

In some embodiments, the computer program product further comprises using at least one of a dichroic mirror and a beam splitter. In some embodiments, said at least one dichroic mirror has a cutoff wavelength selected from the group consisting of 430 nm, 580-660 nm, and 800 nm.

In some embodiments, the computer program product further comprises using confocal imaging means.

In some embodiments, the second light beam has a wavelength selected from the range 495-570 nm, wherein said detecting is further based on detecting blood vessel features below a superficial layer of said tissue sample appearing in at least some of said one or more images.

In some embodiments, the program code further comprises operating a third light beam source for illuminating said tissue sample with a third light beam, wherein each of said second and third light beams has a wavelength selected from the range 585-720 nm, and wherein said detecting is further based on determining a value of oxygen saturation of the blood in said tissue sample.

In some embodiments, the second light beam has a wavelength selected from the range 900-3000 nm, wherein said detecting is further based on determining a value of fluid accumulation in said tissue sample.

In some embodiments, the second light beam has a wavelength selected from the range 100-390 nm, wherein said detecting is further based on measuring fluorescence emitted by one or more excited fluorophores in said tissue sample.

In some embodiments, the program code further comprises operating a projection system configured for illuminating said tissue sample with spatially-structured light, wherein said detecting is further based, at least in part, on depth-resolved measuring of one or more of tissue blood concentration, tissue blood oxygenation, tissue water fraction, tissue perfusion, collagen, lipids, and exogenous agents. In some embodiments, the spatially-structured light is configured for performing spatial frequency domain imaging (SFDI).

In some embodiments, the depth-resolved measuring comprises adjusting at least one of light frequency, wavelength selection, and amplitude modulation of said spatially-structured light.

In some embodiments, the said program code further comprises operating a first polarized light source for producing light beams with at least a first polarization feature, and a second polarized light sources for producing light beams with at least a second polarization feature; and wherein at least some of said plurality of images are being captured during a period for which said first polarized light source is illuminating the tissue sample, and at least some of said plurality of images are being captured during a period for which said second polarized light source is illuminating the tissue sample. In some embodiments, the first and second polarized light sources are configured for illuminating the tissue sample alternately based on one or more predetermined time intervals. In some embodiments, the program code further comprises processing said one or more images to separate light from a superficial single-scattering layer of the tissue sample and light from a deeper diffuse layer of the tissue sample. In some embodiments, the computer program product further comprises using a non-total internal reflection (TIR) birefringent polarizing prism (BPP) optically coupled to the tissue sample and configured for guiding the light waves returning from the tissue sample.

In some embodiments, the computer program product further comprises using one or more optical retarders along the optical path between the first and second polarized light sources and the tissue sample, wherein said one or more optical retarders are configured as a Mueller matrix imaging polarimeter (MMIP) for determining a partial or complete polarization matrix of the image.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1A is a graph showing the absorption coefficient values for hemoglobin;

FIG. 1B illustrates the principles Rayleigh scattering and Mie scattering;

FIG. 1C is a graph showing the scattering coefficient of purple light;

FIG. 2A is a block diagram of an exemplary system for purple light imaging, according to an embodiment;

FIGS. 2B-2E are schematic illustrations of systems for purple light imaging, according to certain embodiments;

FIG. 3 shows the effect of various light wavelengths on depth penetration in a tissue sample;

FIGS. 4A-4B are schematic illustrations of systems for purple light imaging, according to embodiments;

FIG. 5 is a flowchart describing a method for purple light imaging, according to an embodiment; and

FIGS. 6A-6F show experimental results of a system for purple light imaging, according to certain embodiments.

### DETAILED DESCRIPTION

Disclosed herein are a method and a system for using purple light in medical diagnostic procedures, to improve visualization results of certain diagnostic procedures, and thereby enable more accurate detection of certain pathologies. The present disclosure takes advantage of several characteristics of purple light which make it particularly useful in the context of imaging of tissues.

In some embodiments, a method and a system of the present disclosure comprise illuminating a tissue sample in vivo with purple light. In some embodiments, a contrast agent may be further applied to the tissue sample, for enhancing certain features and abnormalities. A detection of tissue abnormality may be based on at least one of color changes in the tissue and blood vessel features appearing in said plurality of images. In some embodiments, one or more imaging devices may be used to capture a plurality of images of the tissue sample, before and/or after the application of the contrast agent.

In some embodiments, the tissue sample is illuminated with purple light in combination with one or more additional lights having specified wavelengths. In such embodiments, tissue abnormalities can further be detected based on one or more of color changes in the tissue sample, blood vessel features appearing at various depths in the tissue sample, fluorescence emitted by the tissue sample, fluid accumulation in the tissue sample, and/or a value of oxygen saturation of the blood in the tissue sample.

A used herein, “purple light” refers to a spectral color in the spectrum of visible light having a dominant wavelength of approximately 390-430 nanometer (nm). Any integer or decimal range of values between 390-430 is also explicitly intended herein. Due to its short wavelength, purple light has the highest spatial resolution of any spectral band within the visible spectrum. However, because it is still part of the visible spectrum, it can be captured by many common imaging devices, such as regular digital RGB (red-green-blue) cameras and monochrome cameras. Purple light also has the highest scattering coefficient (μₛ) of all the visible wavelengths, resulting in a very shallow penetration depth into the tissue, and relatively high amounts of light that is diffusely reflected from the tissue. All of these attributes make purple light particularly beneficial in diagnostic procedures involving visualization through contrast agents that are scattering-based, such as acetic acid. In addition, the short wavelength of purple light makes it useful for exciting fluorescence in some molecules in tissue. For example, it is commonly used to detect bodily fluids, and can also excite fluorescence of extracellular matrix (ECM) molecules, such as collagen and elastin, to help detect changes in the ECM. Additionally, purple light absorption is highly sensitive to levels of hemoglobin in the blood, and therefore may be useful in detecting abnormal vascularization and increased blood content in tissue.

One example of a diagnostic procedure in which purple light may help to improve visualization of results is colposcopy. Colposcopic examination is routinely used as a diagnostic tool for identification of abnormal areas of the cervix. A colposcope functions as a lighted optical magnification device to obtain images providing a general impression of the surface architecture of the cervical tissue, as well as certain vascular patterns that may indicate the presence of more advanced precancerous or cancerous lesions. In the course of colposcopy, acetic acid (typically diluted at 3-5%) is usually applied to the cervix to highlight areas with a high risk of neoplasia, which appear as varying degrees of whiteness, because aceto-whiteness correlates with higher nuclear density. As noted above, purple light may further help to enhance the aceto-whitening effect of the contrast agent.

The following discussion focuses on the use of the present invention in connection with cervical diagnostic procedures. However, in addition to colposcopy, other areas of diagnostic and therapeutic treatments which may benefit from improved visualization in results include, but are not limited to, general surgery (endoscopy and laparoscopy), gastroenterology, ear nose and throat (ENT), urology, forensic medicine (i.e., evidence collection from patients who are criminal victims or suspects), and visualization of injuries in general (including diabetic ulcers).

### Optical Properties of Biological Tissues

For a comprehensive review of optical properties of various biological tissues, see, e.g., S. L. Jacques, “Optical properties of biological tissues: a review”, 2013 Phys. Med. Biol. 58 R37.

Biological tissues can have varying optical properties, which describe the interaction between light and the tissue. These properties are based on absorption and scattering, including reflection, refraction, fluorescence, and others. These properties can be described by optical interaction coefficients, such as the absorption coefficient μₐ; the scattering coefficient μₛ; the anisotropy g; and the reduced scattering coefficient μ′ₛ.

### Optical Absorption

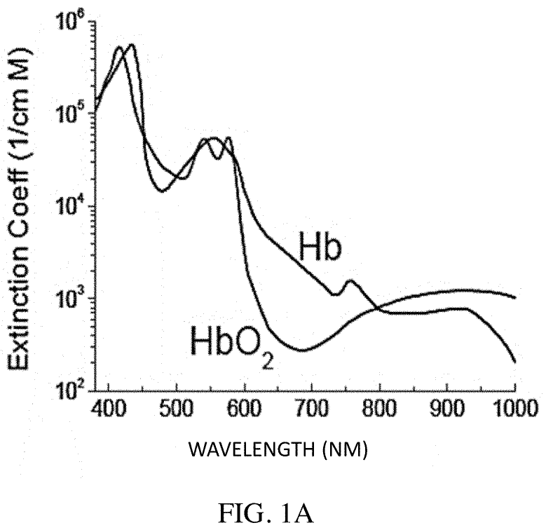

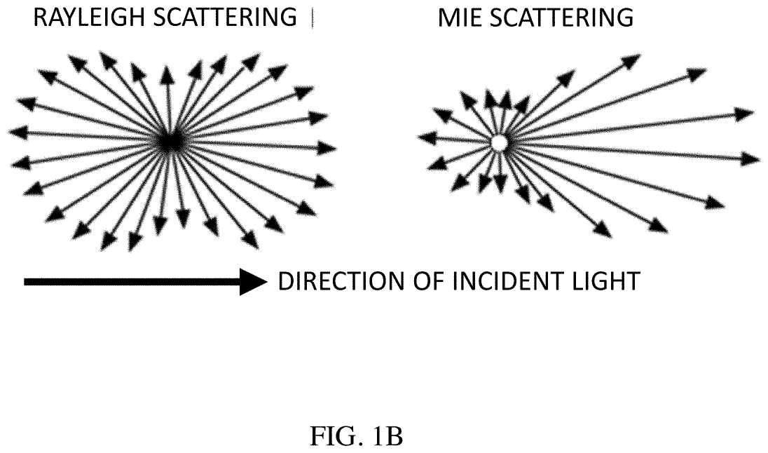

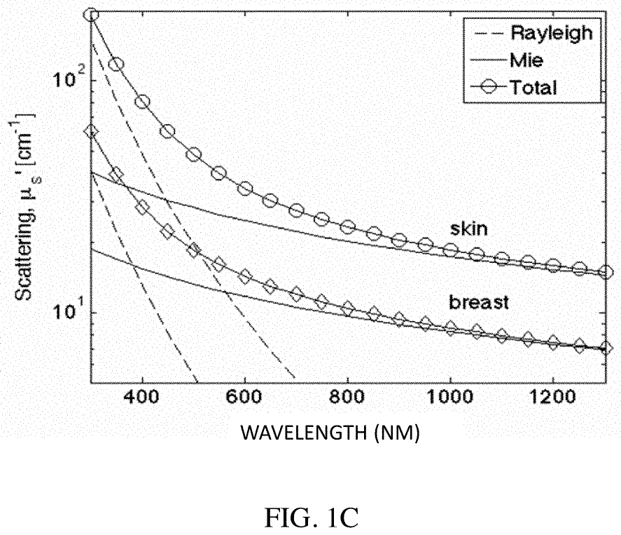

Tissue optical absorption can be described in terms of the fraction of incident light absorbed per incremental length of travel within a tissue. Tissue absorption is determined by the amounts of absorbing chromophores, or light absorbing particles (e.g., blood, water, melanin, fat, yellow pigments) in the tissue. The absorption coefficient pa of a tissue can be wavelength-dependent; for example, chromophores such as hemoglobin (Hb or HGb), lipids and water are the main absorbers in the visible and near-infrared (IR) light ranges. Similarly, protein, amino acids, and DNA dominate ultraviolet (UV) absorption. FIG. 1A shows the absorption coefficient values for hemoglobin, both unbound (deoxy-hemoglobin, deoxy-Hb, or simply Hb) and oxygen-bound (oxy-hemoglobin or HbO₂), as a function of wavelength. Hemoglobin concentration by volume in tissue is generally low (0.2%-2.0% on average for a given volume, up to 15% on average for volume containing a blood vessel) and varies, e.g., among men, women, children, and during pregnancy. Because purple light is highly absorbed by hemoglobin, purple light is sensitive to small changes in the local concentration of hemoglobin. This means that it is possible, for example, to use purple light to identify pathologies in which a biological tissue becomes highly vascularized and/or is bleeding.

### Optical Scattering

Optical scattering is a physical process in which some forms of radiation, such as light, are forced to deviate from a straight trajectory by one or more paths, due to localized non-uniformities in the medium through which they pass. Light scattering arises from the presence of heterogeneities within a bulk medium, such as the distribution of particles with varying refractive indices in the medium. Tissue optical scattering can be described either as scattering by particles that have a refractive index different from the surrounding medium, or as scattering by a medium with a continuous but fluctuating refractive index. Scattering in biological tissues depends on the size, morphology, and structure of the components in tissues (e.g., lipid membrane, collagen fibers, nuclei). Variations in these components due to disease would affect scattering properties, thus providing a potential visual indication for diagnostic purposes.

With reference to FIG. 1B, the terms Rayleigh scattering and Mie scattering are commonly used in the field of biomedical optics. Rayleigh scattering refers to scattering from very small particles, which are generally smaller than the wavelength of light, and is inverse to the wavelength of the incident light. Mie scattering refers to scattering by particles comparable in size to or larger than the wavelength of light. Because of the relative particle size, Mie scattering is said not to be strongly wavelength-dependent and is mostly forward directional scattering.

The anisotropy factor, g, is a measure of the directionality of the scattered light and varies from 0 for isotopically-scattered light to 1 for forwardly-scattered light. The reduced scattering coefficient, μ′ₛ, can be regarded as an effective isotropic scattering coefficient that represent the cumulative effect of several scattering events.

As can be seen in FIG. 1C, purple light (i.e., light having a wavelength between 390-430 nm) has the highest scattering coefficient of all the visible wavelengths (shown with reference to human breast and skin tissue). When considering the reduced scattering coefficient μs′ as a function of wavelength, the purple part of the visible spectrum is where isotropic Rayleigh scattering is at its highest. The results of this are twofold. First, high Rayleigh scattering makes the penetration depth of the light into the tissue very shallow, often under 100 m (micrometers). Second, a relatively large amount of isotropic scattering means that the amount of light that is diffusely reflected from the tissue is higher. All these attributes make purple light particularly sensitive to scattering-based contrast agents on the superficial-most areas of the tissue, such as the acetic acid used in colposcopy. Because acetic acid condenses nuclei normally described by the Mie solution, i.e., larger than the wavelength of light, into smaller particles that follow the Rayleigh approximation, the amount of diffusely reflected light is increased significantly. The result is that aceto-whitened tissue reflects increased amounts of purple light, making the visualization of the procedure more pronounced.

### Purple Light Imaging

Certain exemplary embodiments disclosed herein provide a method and a system for applying purple light to a biological tissue sample in vivo, to improve observation of certain pathologies.

In some embodiments, a system of the present invention illuminates a tissue sample with a purple light beam for observation purposes. A contrast agent may be applied to the tissue sample for enhancing certain features and abnormalities. An imaging device may be used to capture a plurality of images of the tissue sample, wherein a detection of tissue abnormality may be based on at least one of color changes in the tissue, and blood vessel features appearing in said plurality of images.

In some embodiments, the present system comprises various illumination modes, wherein the tissue sample is illuminated with purple light in combination with one or more additional lights transmitted simultaneously or sequentially with the purple light. In some embodiments, any such illumination modes can be selected by a user. In some examples, multiple light sources are provided. In other examples, one or more turrets may be placed in the respective light paths of one or more light source units, and serve to insert and/or withdraw from the light paths multiple optical filters having different spectral transmittance properties, for selectively passing or rejecting passage of radiation in a wavelength-, polarization-, and/or frequency-dependent manner. In yet other examples, rotary filter wheels may be placed in the light paths of the light sources, which rotary filter wheels serve to sequentially insert and/or withdraw optical filters in the light paths within a rotation cycle of the rotary filter wheels equipped with optical filters having different spectral transmittance.

The various illumination modes of the present system may be used independently or in conjunction with one another within a single medical procedure. For example, one illumination mode may be used to identify the location of an abnormality or lesion. Once the location and rough range of the lesion becomes clear, a second illumination mode may be used for examining blood vessel structure at various depths from the surface of the tissue, as well as for clarifying the boundary between a lesion tissue and normal tissue. Finally, a third illumination mode may be used for ascertaining the infiltration of the lesion into the tissue.

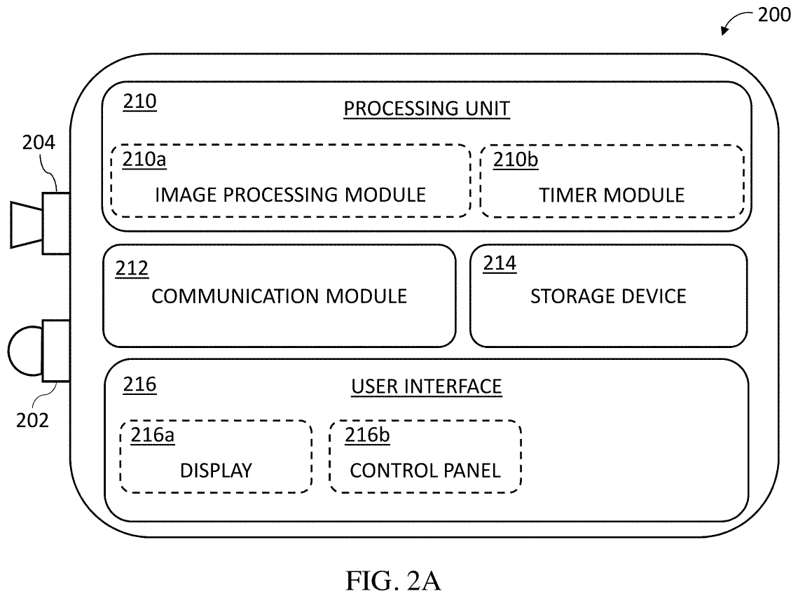

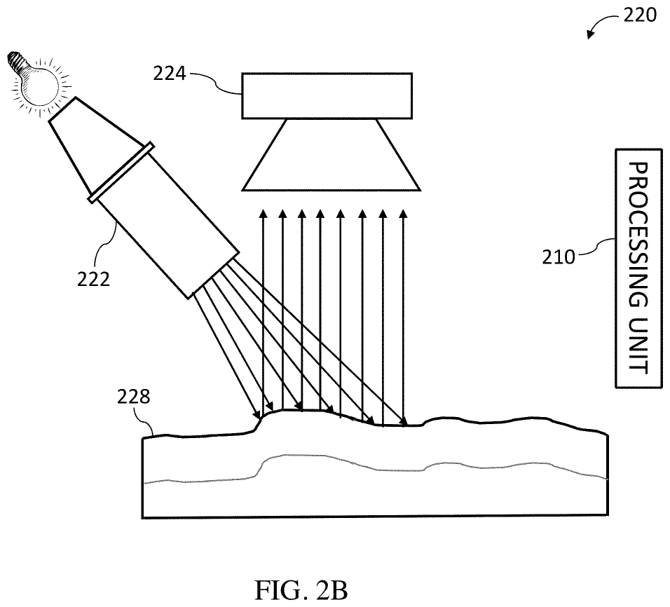

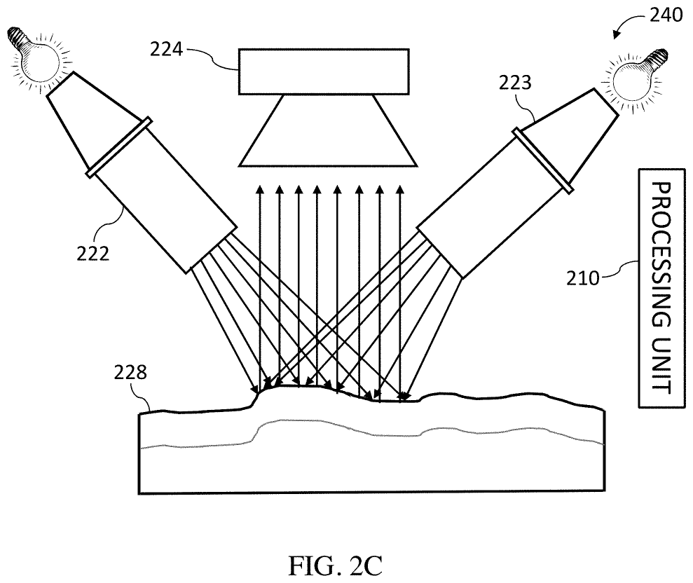

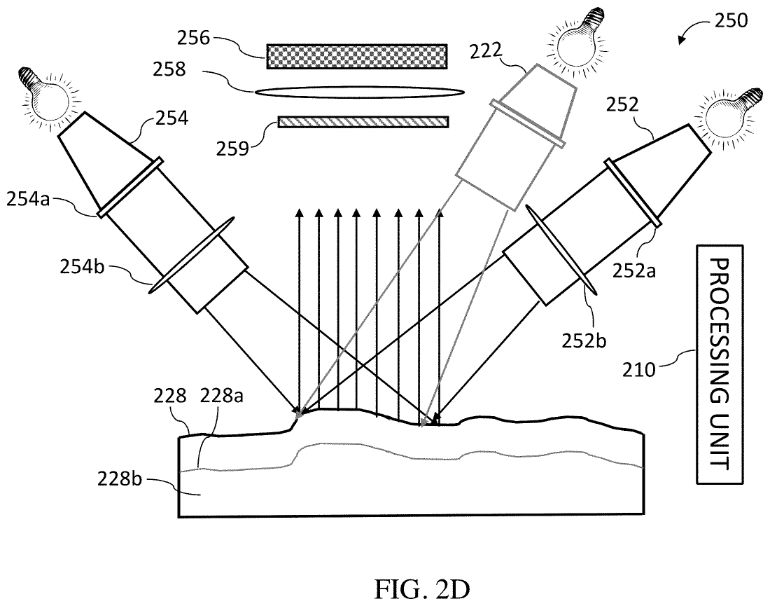

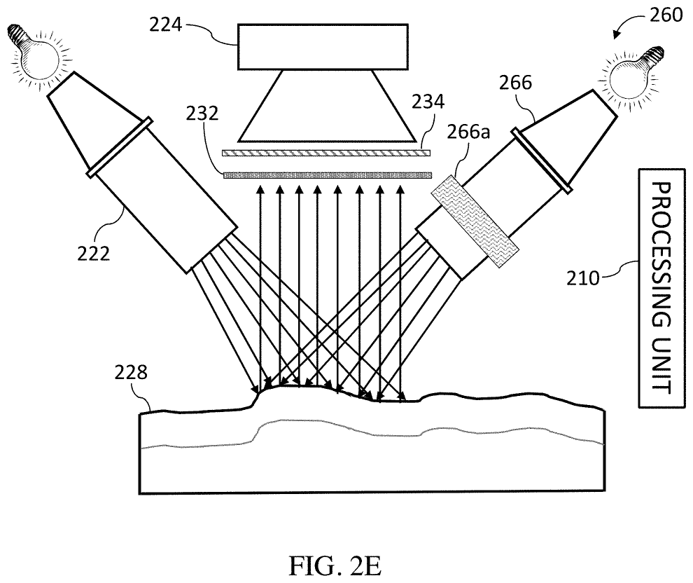

FIG. 2A schematically illustrates an exemplary system 200 according to the present disclosure. System 200 comprises a light source 202, an imaging device 204, and a processing unit 210. According to an embodiment, light source 202 is configured to produce purple light, wherein a light beam produced by light source 202 has a wavelength selected from the range 390-430 nanometer (nm). Any integer or decimal range of values between 390-430 is also explicitly intended herein. Light source 202 may comprise, e.g., any suitable light source selected from the group consisting of incandescent light bulb, light emitting diode (LED), laser diode, light emitter, bi-spectral emitter, dual spectral emitter, photodiode, and semiconductor die. In some embodiments, light source 202 is configured to illuminate the tissue sample directly. In other embodiments, light source 202 is configured to transmit illumination through a suitable conduit, e.g., an optical fiber cable, for higher focus and intensity of illumination.

In some embodiments, imaging device 204 may be configured to detect RGB (red-green-blue) spectral data. In other embodiments, imaging device 204 may be configured to detect at least one of monochrome, ultraviolet (UV), near infrared (NIR), and short-wave infrared (SWIR) spectral data. In some embodiments, imaging device 204 comprises a digital imaging sensor selected from the group consisting of complementary metal-oxide-semiconductor (CMOS), charge-coupled device (CCD), Indium gallium arsenide (InGaAs), and polarization-sensitive sensor element. In some embodiments, imaging device 204 is configured to capture images of the tissue sample along a direct optical path from the tissue sample. In other embodiments, imaging device 204 is coupled to a light guide, e.g., a fiber optic light guide, for directing a reflectance and/or fluorescence from the tissue sample to imaging device 204.

Imaging device 204 may further comprise, e.g., zoom, magnification, and/or focus capabilities. Imaging device 204 may also comprise such functionalities as color filtering, polarization, and/or glare removal, for optimum visualization. Imaging device 204 may include an image stream recording system configured to receive and store a recording of an image stream received, processed, and/or presented through system 200.

In some embodiments, imaging device 204 may be configured to capture a plurality of RGB images, wherein imaging device 204 and/or processing unit 210 may be configured to change the ratio of individual RGB channels, for example, by amplifying at least one of the green channel and the red channel. In such embodiments, the detection of tissue abnormalities in the tissue sample can be based, at least in part, on detecting fluorescence emitted by sample 202.

System 200 may store in a non-volatile memory thereof, such as storage device 214, software instructions or components configured to operate a processing unit (also referred to as a “hardware processor,” “CPU,” or simply “processor”), such as processing unit 210. In some embodiments, the software components may include an operating system, including various software components and/or drivers for controlling and managing general system tasks (e.g., memory management, storage device control, power management, etc.) and facilitating communication between various hardware and software components. The software instructions and/or components operating processing unit 210 may include instructions for receiving and analyzing multiple frames captured by imaging device 204. For example, processing unit 210 may comprise image processing module 210a, which receives one or more live image streams from imaging device 204 and applies one or more image stream processing algorithms to the received image streams. In some embodiments, image processing module 210a comprises one or more algorithms configured to perform, e.g., object recognition and classification in images captured by imaging device 204, using any suitable image processing or feature extraction technique. For some embodiments, image processing module 210a can simultaneously receive and switch between multiple input image streams to multiple output devices while providing image stream processing functions on the image streams. The incoming image streams may come from various medical or other imaging devices. The image streams received by the image processing module 210a may vary in resolution, frame rate (e.g., between 15 and 35 frames per second), format, and protocol according to the characteristics and purpose of their respective source device. Depending on the embodiment, the image processing module 210a can route image streams through various processing functions, or to an output circuit that sends the processed image stream for presentation, e.g., on a display 216a, to a recording system, across a network, or to another logical destination. In image processing module 210a, the image stream processing algorithm may improve the visibility and reduce or eliminate distortion, glare, or other undesirable effects in the image stream provided by an imaging device. An image stream processing algorithm may reduce or remove fog, smoke, contaminants, or other obscurities present in the image stream. The types of image stream processing algorithms employed by the image stream processing module 210a may include, for example, a histogram equalization algorithm to improve image contrast, an algorithm including a convolution kernel that improves image clarity, and a color isolation algorithm. The image stream processing module 210a may apply image stream processing algorithms alone or in combination. In some embodiments, Image processing module 210a is configured to perform f RGB channel separation and amplification.

Image processing module 210a may also facilitate logging or recording operations with respect to an image stream from imaging device 204. According to some embodiments, image processing module 210a enables recording of the image stream with a voice-over, bookmarks, and/or capturing of frames from an image stream (e.g., drag-and-drop a frame from the image stream to a window). Some or all of the functionality of the image processing module 210a may be facilitated through an image stream recording system or an image stream processing system.

Processing unit 210 may also comprise timer module 210b, which may provide countdown capabilities using one or more countdown timers, clocks, stop-watches, alarms, and/or the like, that trigger various functions of system 200, such as image capture. Such timers, stop-watches and clocks may also be added and displayed over the image stream through user interface 216. For example, the timer module 210b may allow a user to add a countdown timer, e.g., in association with a surgical or diagnostic and/or other procedure. A user may be able to select from a list of pre-defined countdown timers, which may have been pre-defined by the user. In some variations, a countdown timer may be displayed on a display 216a, bordering or overlaying the image stream.

In some embodiments, system 200 comprises a communication module (or set of instructions), a contact/motion module (or set of instructions), a graphics module (or set of instructions), a text input module (or set of instructions), a Global Positioning System (GPS) module (or set of instructions), voice recognition and/or and voice replication module (or set of instructions), and one or more applications (or set of instructions). For example, a communication module 212 may connect system 200 to a network, such as the Internet, a local area network, a wide area network and/or a wireless network. Communication module 212 facilitates communication with other devices over one or more external ports, and also includes various software components for handling data received by system 200. For example, communication module 212 may provide access to a patient medical records database, e.g., from a hospital network. The content of the patient medical records may comprise a variety of formats, including images, audio, video, and text (e.g., documents). In some embodiments, system 200 may access information from a patient medical record database and provide such information through the user interface 216, presented over the image stream on display 216a. Communication module 212 may also connect to a printing system configured to generate hard copies of images captured from an image stream received, processed, or presented through system 200.

In some embodiments, storage device 214 (which may include one or more computer readable storage mediums) of system 200 is used for storing, retrieving, comparing, and/or annotating captured frames. Image frames may be stored on storage device 214 based on one or more attributes, or tags, such as a time stamp, a user-entered label, or the result of an applied image processing method indicating the association of the frames, to name a few.

In some embodiments, a user interface 216 of system 200 comprises a display monitor 216a for displaying images, a control panel 216b for controlling system 200, and a speaker 216c for providing audio feedback. In some variations, display 216a may be used as a viewfinder and/or a live display for either still and/or video image acquisition by imaging device 204. The image stream presented by display 216a may be one originating from imaging device 204. Display 216a may be a touch-sensitive display. The touch-sensitive display is sometimes called a “touch screen” for convenience and may also be known as or called a touch-sensitive display system. Touch-sensitive display may be configured to detect commands relating to activating or deactivating particular functions of system 200. Such functions may include, without limitation, image stream enhancement, management of windows for window-based functions, timers (e.g., clocks, countdown timers, and time-based alarms), tagging and tag tracking, image stream logging, performing measurements, two-dimensional to three-dimensional content conversion, and similarity searches.

In some embodiments, control panel 216b includes one or more user input control devices, such as a physical or virtual joystick, mouse, and/or click wheel. In other variations, system 200 comprises one or more of a peripherals interface, RF circuitry, audio circuitry, a microphone, an input/output (I/O) subsystem, other input or control devices, optical or other sensors, and an external port. System 200 may also comprise one or more sensors, such as proximity sensors and/or accelerometers. Each of the above identified modules and applications corresponds to a set of instructions for performing one or more functions described above. These modules (i.e., sets of instructions) need not be implemented as separate software programs, procedures or modules, and thus various subsets of these modules may be combined or otherwise re-arranged in various embodiments.

In some embodiments, system 200 is mounted on a stand, a tripod and/or amount, which may be configured for easy movability and maneuvering (e.g., through the use of caster wheels). In some embodiments, the stand may incorporate a swing arm. In such embodiments, light source 202 and/or imaging device 204 may be mounted on the swingarm, to allow hands-free, stable positioning and orientation of imaging device 204 for desired image acquisition. In other embodiments, system 200 is a portable, hand-held system.

System 200 described herein is only an exemplary embodiment of the present disclosure, and may have more or fewer components than shown, may combine two or more components, or a may have a different configuration or arrangement of the components. The various components of system 200 may be implemented in hardware, software or a combination of both hardware and software, including one or more signal processing and/or application-specific integrated circuits. In various embodiments, system 200 may comprise a dedicated hardware device, or may form an addition to or extension of an existing medical device, such as a colposcope. According to various other embodiments, the processing unit 220 or processing tasks performed thereby may be implemented by a handheld or worn computing device such as, but not limited to, a smart phone, a tablet computer, a notepad computer, and the like. In addition, aspects of the present system which can be implemented by computer program instructions, may be executed on a general-purpose computer, a special-purpose computer, or other programmable data processing apparatus.

Reference is made to FIG. 2B, which schematically shows a system 220 in operation, according to certain embodiments. A light source 222 illuminates a tissue sample 228 under observation. Light source 222 irradiates tissue sample 228 using slight in the purple wavelength region, which only reaches depths very near to the surface of tissue sample 228. By utilizing this fact, visual information specifically concerning the mucosa surface layer of tissue sample 228 can be obtained.

An imaging device 224 is used to capture one or more images of tissue sample 228. In some embodiments, the visibility of abnormalities and blood vessel features in tissue sample 228 may be enhanced through the application of a suitable contrast agent, such as diluted acetic acid, Lugol's iodine, and/or another agent. In some embodiments, detecting tissue abnormalities in sample 202 is based, at least in part, on color changes and/or blood vessel features appearing in sample 202. In some embodiments, at least some of the images captured by imaging device 224 are being captured before the step of applying a contrast agent, and at least some of the images are being captured after the step of applying a contrast agent. In such embodiments, the detection of tissue abnormalities is based, at least in part, on a comparison between the images taken before and after applying the contrast agent. In other embodiments, at least some of the images are being captured at a specified time period after the step of applying the contrast agent. For example, in the case of a contrast agent which is acetic acid, the specified time period can be between 1 and 600 seconds. Any integer or decimal value between 1 and 600 is also explicitly intended herein. The images captured by imaging device 204 are being sent to processing unit 210 for processing, as described above.

FIG. 2C shows a system 240 according to certain embodiments. System 240 comprises at least two light sources 222, 223 configured for illuminating tissue sample 228. In some embodiments, light source 222 emits purple light, whereas light source 223 emits a light beam having a different wavelength. In some embodiments, light sources 222, 223 are configured to illuminate tissue sample 228 simultaneously, wherein imaging device 224 is configured to capture images of tissue sample 228 being illuminated by both of light sources 222, 223 at the same time. In other embodiments, light sources 222, 223 are configured to illuminate tissue sample 228 sequentially. In such embodiments, at least some images of tissue sample 228 are being captured by imaging device 224 during a period for which light source 222 is illuminating tissue sample 228, and at least some images during a period for which light source 223 is illuminating tissue sample 228.

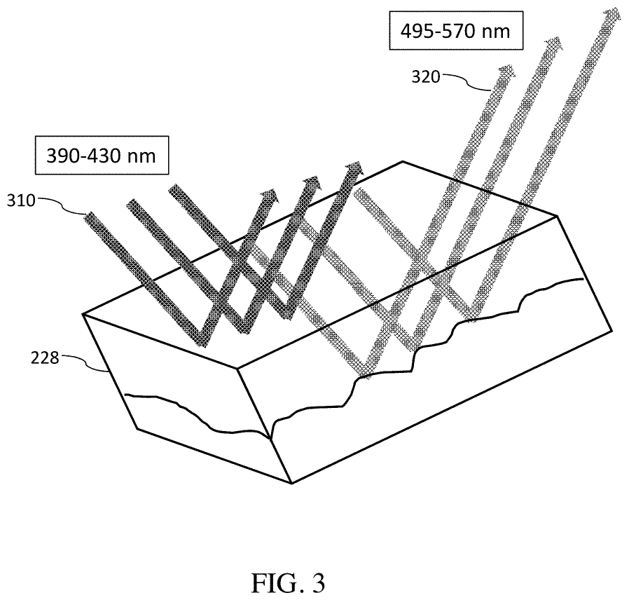

In some embodiments, light source 223 has a wavelength selected from the range 490-580 nm (i.e., the green wavelength region). Any integer or decimal range of values between 490-580 is also explicitly intended herein. As shown in FIG. 3, light beam 320 having wavelengths in the 490-580 nm reaches a depth that is a little deeper than the surface layer of tissue sample 228. This is in contrast to light beam 310, which only reaches depths very near to the surface of tissue sample 228. Accordingly, in such embodiments the detection of abnormalities in tissue sample 228 is based, at least in part, on enhancing the resolution of blood vessel features below a superficial layer of tissue sample 228.

In some embodiments, light source 223 comprises one or more light beams each having a wavelength selected from the range 585-720 nm (i.e., the red wavelength region). Any integer or decimal range of values between 585-720 is also explicitly intended herein. In such embodiments, the detection of abnormalities in tissue sample 228 is based, at least in part, on determining a value of oxygen saturation of the blood in said tissue sample.

In some embodiments, light source 223 has a wavelength selected from the range 900-3000 nm (i.e., the near-infrared and shortwave infrared wavelength regions). Any integer or decimal range of values between 900-3000 is also explicitly intended herein. In such embodiments, the detection of abnormalities in tissue sample 228 is based, at least in part, on determining a value of fluid accumulation in tissue sample 228.

In some embodiments, light source 223 has a wavelength selected from the range 100-390 nm (i.e., the ultraviolet wavelength region). Any integer or decimal range of values between 100-390 is also explicitly intended herein. In such embodiments, the detection of abnormalities in tissue sample 228 is based, at least in part, on measuring fluorescence emitted by one or more excited fluorophores in tissue sample 228.

In some embodiments, there are multiple lights sources having different wavelengths, such as the wavelengths described above.

FIG. 2D depicts an exemplary schematic block diagram of a system 250 according to an embodiment of the present invention, which may comprise purple light illumination in combination with polarization difference imaging (PDI).

PDI may be used for capturing a plurality of images of a sample, to determine a spatial difference of the light intensity by comparing one frame of the sample to another. For example, when looking for a sample with a superficial structure in its single-scattering layer, the light returning from deeper structures can drown out light from a layer of interest. This drowning-out occurs because most of the light returning from the sample (for example, 80% of the reflected light in skin) is diffuse. In addition, there is a specular reflection dependent on a refractive index of the sample and the angular extent of the illumination. Such specular reflection makes up roughly 15% of the reflected light. The layer of interest in the superficial single-scattering layer thus makes up only about 4-5% of the reflected light. Removing this background signal allows for highlighting the layer of interest in the superficial structure. Eliminating the background signal is thus the key principle of PDI systems as a contrast enhancement mechanism.

System 250 includes a plurality of light sources such as light sources 252, 254; a plurality of illumination optics, such as 252a, 254a; a plurality of linear polarizers, such as 252b, 254b and linear polarizer 259; a detection optic unit 258, and imaging sensor array 256. In some embodiments, system 250 may further comprise a purple light source 222.

Each light source 252, 254 is equipped with a polarization separating mechanism. Therefore, each light source 252, 254 is configured to produce light beams with a unique polarization towards a sample 228. Each light source 252, 254 is coupled to an illumination optic 252a, 254a, respectively. Each illumination optic 252a, 254a is used to guide the light beams transmitted from each light source 252, 254 toward sample 228. In addition, each illumination optic 252a, 254a is coupled to one of the plurality of linear polarizers, e.g., 254a or 254b. Each of the linear polarizers 254a and 254b is configured to produce a linearly polarized light respective of the light beams transmitted from the respective light source 252, 254 and guided by the illumination optic 252a, 254a.

In an embodiment, system 250 includes an additional linear polarizer 259 coupled to the detection optic unit 258. Such linear polarizer 259 is configured to transmit light that is linearly polarized (e.g., within orientation of 180°) or, alternatively, circularly polarized light to the detection optic unit 258. Then the detection optic unit 258 guides the polarized light towards the sensor array 256.

The sensor array 256 is configured to capture a plurality of frames of the sample 228. Each of the frames is captured respective of a unique polarization of the light sources 252, 254, either by coordinating the illumination according to a predetermined time interval or by distinguishing between polarization states based on predetermined markers interspersed between the unique polarization states. The captured frames are analyzed under the control of processing unit 210 to produce an output image that represents the difference between the various polarizations. In some embodiments, system 250 may be utilized for separating light from a superficial single-scattering layer 228a of tissue sample 228, and its deeper diffuse layer 228b, as a function of space. According to some embodiments, the processing unit 210 is configured to produce an output image showing the differences between various polarizations when a plurality of frames is captured as a function of time.

Certain embodiments of system 250 may further comprise a non-total internal reflection (TIR) birefringent polarizing prism (BPP) to maximize a refraction difference between ordinary waves and extraordinary waves of light returning from the sample; and a detection optic unit coupled to the non-TIR BPP for guiding the light waves returning from the sample towards a single-polarization sensitive imaging sensor array configured to capture at least one frame of the sample respective of the light waves returning from the superficial single-scattering layer of the sample apart, from the deeper diffuse layer.

With reference to FIG. 2E, in some embodiments, a system 260 according to an embodiment may comprise structured illumination techniques, such as Spatial Frequency Domain Imaging (SFDI). As noted above, the tissue components known as chromophores (such as oxy-hemoglobin, deoxy-hemoglobin, and water) can be detected optically to assess various indicators or indices of local tissue health or physiological status. Examples of such indices include tissue oxygen saturation (stO₂, or fraction of oxygenated blood), total blood volume (ctTHb), tissue water fraction (ctH₂O), and tissue perfusion or metabolism. Chromophores can be detected because they have absorption spectra with detectable features, in the visible and/or near infrared regions, such that a light source can be used to illuminate a tissue sample, and the remitted light can be used to measure the absorption features in tissue and quantify the chromophore of interest. In practice, however, the presence of scattering in tissue can make this measurement more difficult.

Structured illumination can be employed to facilitate this process. Structured illumination comprises illumination of a tissue sample with one or more spatially structured intensity patterns over a large area of the tissue, and collecting and analyzing the resulting light received back from the sample. An analysis of the amplitude and/or phase of the spatially-structured light received back from the sample as a function of spatial frequency or periodicity, often referred to as the modulation transfer function (MTF), can be used to determine the sample's optical property information at specific wavelengths, including light absorption, light scattering (magnitude and/or angular-dependence), and light fluorescence. Analysis of this light-dependent data can be used to generate images or maps of the area under observation which separate absorption (μₐ) and fluorescence (μₐ) from scattering effects. Structured illumination thus enables quantitative spectroscopy of tissue chromophores and derived physiology parameters (such as tissue oxygen saturation and blood volume, stO₂ and ctTHb). For a further review of structured illumination methods, see, e.g., Cuccia, D. J., “Quantitation and mapping of tissue optical properties”, Journal of Biomedical Optics 14_2_, 024012_March/April 2009; Cuccia, D. J. et al., “Modulated imaging: quantitative analysis and tomography of turbid media in the spatial-frequency domain using modulated imaging”, Optics Letters/Vol. 30, No. 11/Jun. 1, 2005.

With continues reference to FIG. 2E, in some embodiments, system 260 of the present invention may comprise structured illumination methods, such as SFDI. SFDI is capable of quantifying wide-field subsurface optical properties, which can then be utilized to quantify chromophore concentrations for in vivo tissue. In SFDI systems, during imaging, spatially modulated illuminations are projected onto the region of interest over a range of wavelengths. Diffusely reflected light is recorded using, e.g., an imaging device comprising a CCD sensor, and then demodulated in order to extract the diffuse reflectance at each wavelength and spatial frequency, which can then be further reduced into absorption (μₐ) and reduced scattering (μ′ₛ) coefficients by fitting to a known forward model. In some embodiments, system 260 comprising SFDI has the ability to interrogate skin depths of between 1 to 5 mm, to measure spatially-resolved concentrations of chromophores. In some embodiments, imaging penetration depth may be a function, at least in part, of the spatial frequency of illumination. Accordingly, varying the spatial frequency of the illumination pattern allows of controlled depth sensitivity.

In some embodiments, system 260 comprises SFDI source 266, which may be used to generate spatially-modulated illuminations by employing a spatial light modulator (SLM) 266a. SLM 266a may comprise, e.g., a digital micromirror. A bandpass filter 232 for wavelength selection may be used in conjunction with imaging device 224. In some embodiments, crossed linear polarizers, such as polarizer 234, can be added to further select the diffuse reflectance, especially when observing surfaces where specular light can be reflected at all angles.

In some embodiments, system 260 may comprise a purple light source 222. When combined purple with SFDI source 266, purple light source 222 may be used to acquire superficial-level information, e.g., from the outer epithelium), while SFDI source 266 may acquire depth-resolved information at larger imaging depths (e.g., from the stroma, which also has the underlying blood supply to the epithelium). In such cases, differences in blood supply could suggest pathology.

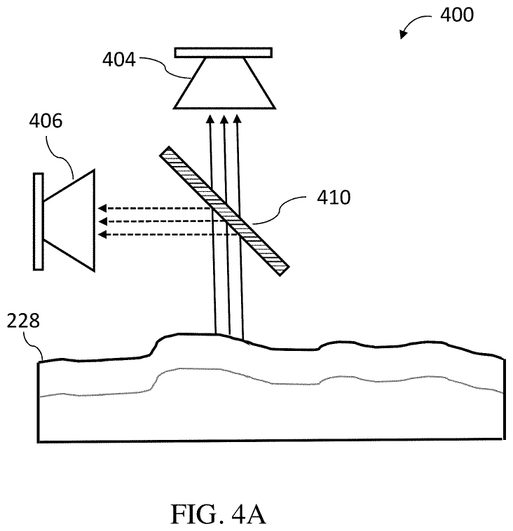

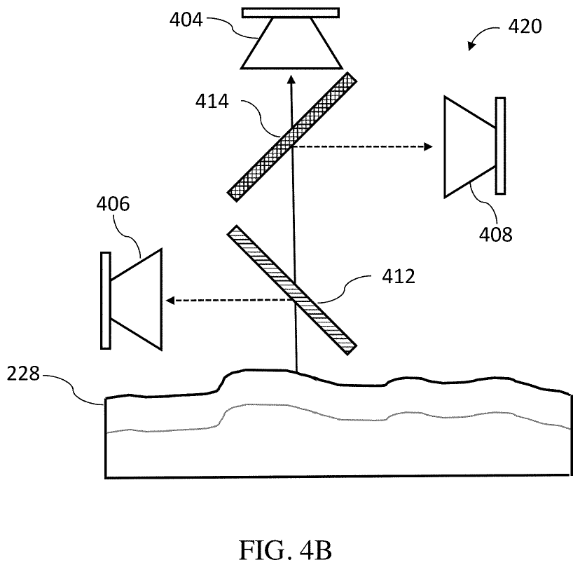

In some embodiments, a system of the present disclosure may comprise more than one imaging device to capture different types of images. Such embodiments may further comprise optical elements such as beam splitters and dichroic mirrors, to split and direct a desired portion of the spectral information emanating from the sample tissue towards each imaging device. With reference to FIG. 4A, a system 400 comprises two imaging devices 404, 406 configured to capture different spectral bands. For example, imaging device 404 may be configured to capture RGB images, and imaging device 406 may be configured to capture monochrome images. The optical path between tissue sample 228 and imaging devices 404, 406 comprises, e.g., a dichroic mirror 410 which is an optical element that passes a first portion of a radiation beam and reflects a second portion of the beam. For example a dichroic mirror can be configured to selectively pass radiation in a first wavelength range and reflect radiation in a second, different radiation range. Accordingly, dichroic mirror 410 may be configured to transmit only blue light towards imaging device 404, and reflect green and red light towards imaging device 406. In some embodiments, the dichroic mirror has a cutoff wavelength selected from the group consisting of 430 nm, 580-660 nm, and 800 nm.

With reference to FIG. 4B, a system 420 comprises three imaging devices 404, 406, 408. System 420 further comprises a beam splitter 412 and a dichroic mirror 414. Beam splitter 412 may split the white light emanating from tissue sample 228 in two parts directed towards imaging device 404 and dichroic mirror 414, respectively. Dichroic mirror 414 in turn transmits blue light towards imaging device 404, and reflects green and red light towards imaging device 408. Thus, each of the imaging devices 404, 406, 408 receives a different spectral portion of the light emanating from tissue sample 228. In other embodiments, similar various configurations may comprise a plurality of imaging devices and a plurality of optical elements including, but not limited to, dichroic mirrors, beam splitters, confocal imaging, and the like.

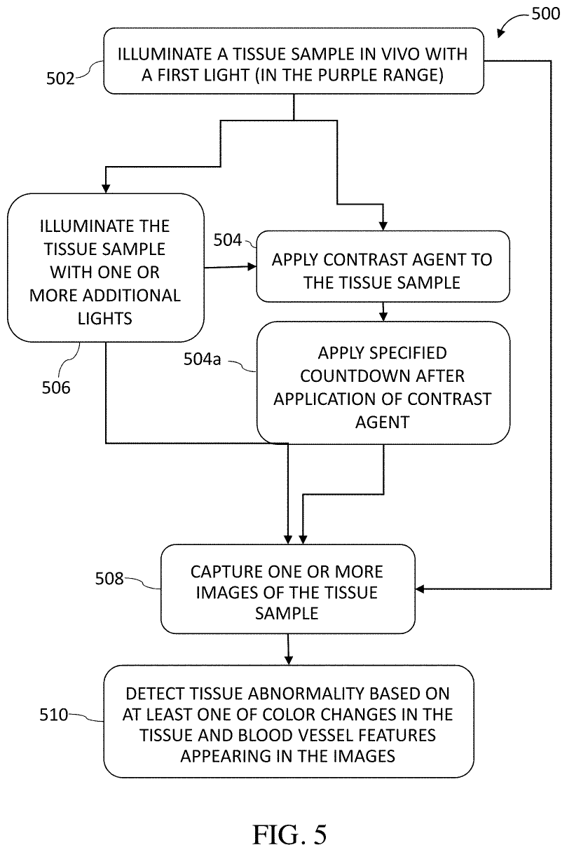

FIG. 5 shows an exemplary flowchart 500 describing a method of the present disclosure. In a step 502, a tissue sample is being illuminated with a purple light. In a step 504, a contrast agent is applied to the tissue sample. In a step 508, images of the tissue sample are captured using an imaging device. In some embodiments, at least some of the images are being captured before step 504 of applying a contrast agent, and at least some of the images are being captured after step 504 of applying a contrast agent. In some embodiments, an optional step 504a provides for capturing at least some of the images at a specified time period after step 504 of applying the contrast agent. Optionally, in a step 506, the tissue sample may be illuminated with one or more additional lights. In some embodiments, the two or more light sources illuminate the tissue simultaneously, wherein images of the tissue sample are being captured in step 508 while it is being illuminated by the one or more light sources at the same time. In other embodiments, the one or more light sources are configured to illuminate the tissue sample sequentially. In such embodiments, at least some images of tissue sample are being captured in step 508 during a period for which only the purple light source is illuminating the tissue sample, and at least some images during a period for which one or more other light sources are is illuminating the tissue sample. In a step 510, the images captured in step 508 are analyzed to detect tissue abnormality based upon one or more visual features of the images, including, but not limited to, one or more of color changes in the tissue sample, blood vessel features appearing at various depths in the tissue sample, fluorescence emitted by the tissue sample, fluid accumulation in the tissue sample, and a value of oxygen saturation of the blood in the tissue sample.

Every functionality of the system described above is explicitly intended herein as a step or a sub-step of the present method. Similarly, every step or sub-step of the method described above is intended to be a functionality of the present system.

In some of the embodiments of the method and the system, thermal imaging may be used in conjunction with the imaging under purple illumination, in order, for example, to detect inflammation. As inflamed tissue does not normally reflect much of the purple light, both in the area with increased blood flow, and in the surrounding area where matrix metalloproteinases digest the extracellular (collagen) matrix decreasing the backscattered signal. Thus, adding thermal imaging to the method and a thermal imager to the system may improve their ability to detect inflammation in the imaged tissue, e.g., the cervix by enhancing contrast. Inflamed tissue may appear warmer in the thermal imagery than normal tissue, e.g., by at least 0.2° C., 0.4° C., 0.6° C., 0.8° C., or more. Accordingly, if a certain area in the imaged tissue (e.g., the cervix) is warmer than its surroundings over the aforementioned threshold, the present system and method may automatically indicate to the user that the certain area is potentially inflamed.

In some of the embodiments of the method and the system, multiphoton imaging (also referred to as ‘multiphoton microscopy’ and ‘multiphoton excitation microscopy’) may be used in conjunction with the imaging under purple illumination, in order, for example, to image deeper structures in the tissue. As purple light imaging typically examines only the superficial tissue, combining it with multiphoton imaging may yield imagery of both the surface and the deeper contents of the imaged tissue. Multiphoton imaging focuses photons from higher wavelengths at larger depths, where they combine to excite fluorescence. The combined photons act as a single photon of lower wavelengths, but penetrate into larger depths because they started at higher wavelengths. Typically, the wavelength they combine into is in the purple-blue range of the spectrum. Accordingly, adding multiphoton imaging to the method and a multiphoton imager to the system may yield three-dimensional imagery of the tissue, e.g., the cervix.

In some of the embodiments of the method and the system, second-harmonic imaging microscopy (SHIM) (which is based on a nonlinear optical effect known as second-harmonic generation (SHG)) may be used in conjunction with the imaging under purple illumination, in order, for example, to enhance the contrast in the purple light imagery and thus more easily detect various structures of interest in the tissue, e.g., the cervix. The essence of SHIM is that certain molecular components in tissue reflect frequency that is exactly double the frequency of light impinging on those molecular components. Accordingly, adding SHIM to the method and a SHIM apparatus to the system may yield contrast-enhanced imagery of the tissue, e.g., the cervix.

In some embodiments of the method and system, the one or more light beams (e.g., purple, green, red, NIR, SWIR, UV, etc.) are steered across the tissue (e.g., the cervix) to illuminate different areas within the field of view of the camera capturing the images. Multiple images may be captures, each with illumination directed to a different area, and the images stitched together using known techniques to yield a composite image of the entire tissue within the camera's field of view.

In some of the embodiments of the method and the system, functional Near-Infrared Spectroscopy (fNRS) may be used in conjunction with the imaging under purple illumination, in order, for example, to perform oximetry of the tissue by detecting hemodynamic responses of the tissue associated with, e.g., cervical tissue. Accordingly, adding fNIRS to the method and an fNIRS apparatus to the system may yield oximetric measurements of tissue, e.g., the cervix.

### Experimental Results

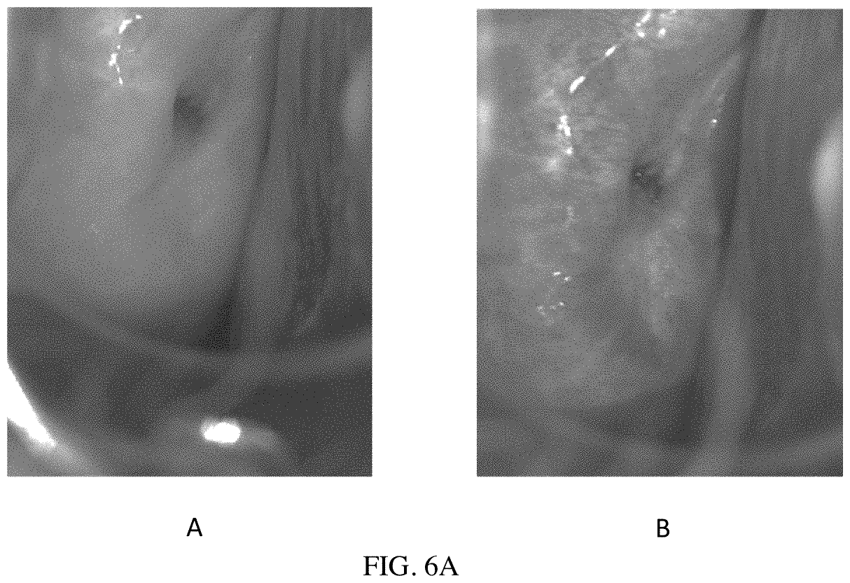

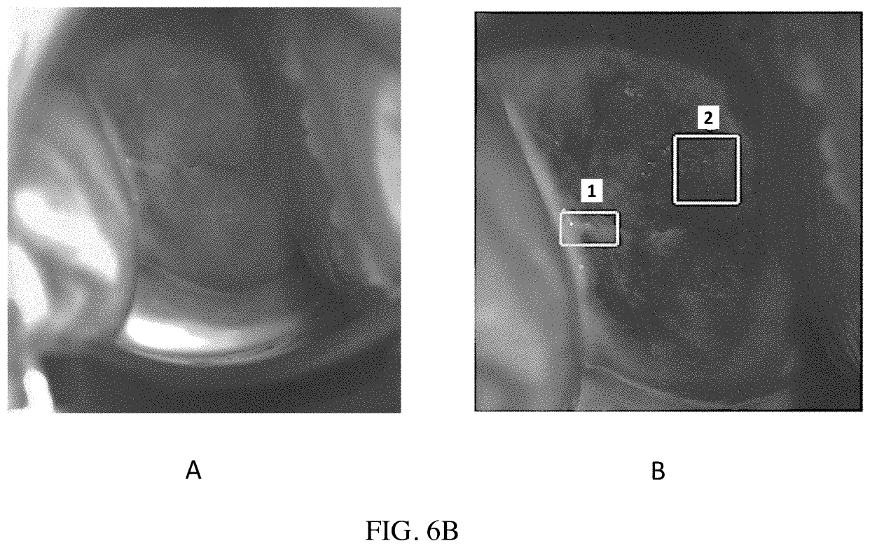

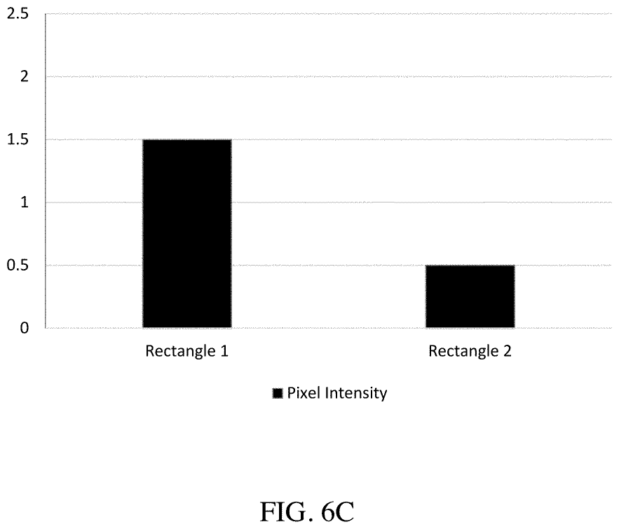

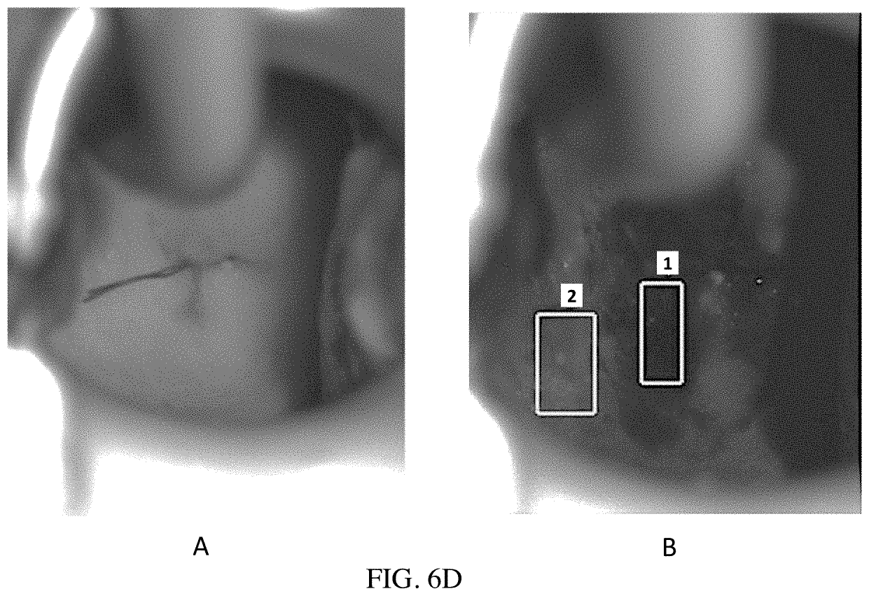

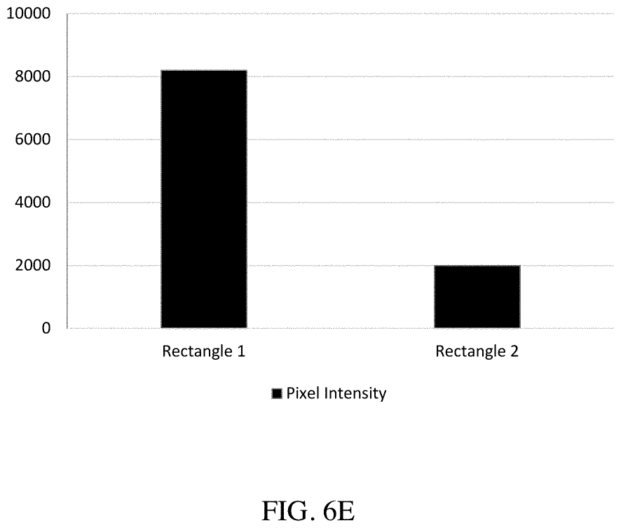

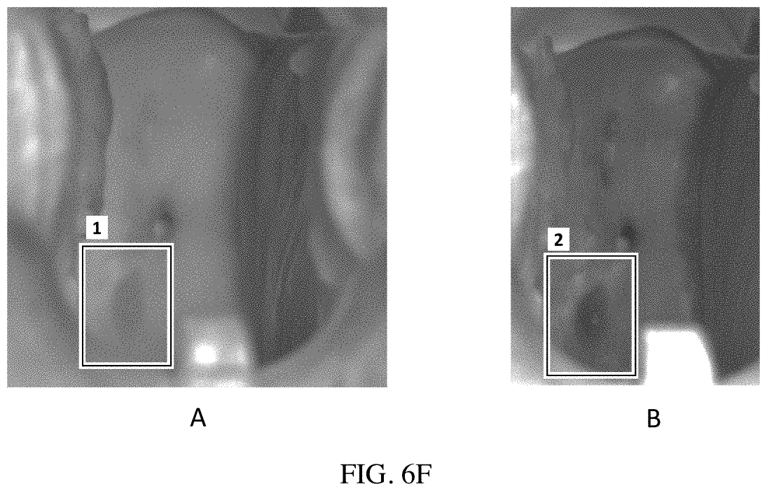

FIGS. 6A-6F show examples of images and analysis results of colposcopy imaging, as compared to regular RGB imaging. The images illustrate the ability of purple light to highlight additional structures, particularly aceto-whitened regions and highly vascularized regions.

FIG. 6A depicts the cervix of a patient with low grade dysplasia (LSIL). Additional structures close to the surface can be seen in the image taken under purple light (panel B), but are not as clearly visible in panel A.

FIG. 6B depicts the cervix of a patient with CIN 3 scheduled for a loop electrosurgical excision procedure (LEEP). Acetowhitening is seen in both RGB (panel A) and purple light (panel B). As shown in the graph in FIG. 6C, the aceto-whitened areas (e.g., rectangle 1 in panel B, FIG. 6B) shows greater pixel intensity than surrounding tissue (e.g., rectangle 2, panel B, FIG. 6B).

FIG. 6D shows the cervix of a patient with dysplasia. Higher blood content can be seen in the purple image as dark areas (panel B), but not the RGB image (panel A). As shown in the graph in FIG. 6E, rectangle 1 in panel B shows greater pixel intensity than surrounding tissue in rectangle 2.

FIG. 6F shows the cervix of a patient scheduled for a LEEP procedure with both a vascular and aceto-whitening abnormality. A vascular lesion (rectangles 1, 2) can be seen in both the RGB image (panel A) and the purple light image (panel B), with an aceto-whitened region to the left. However, both the vascular and aceto-whitened areas can be with greater visibility and contrast in the purple light image (panel B).

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1-30. (canceled)

31. A system comprising:
a light beam source;
an imaging device;
at least one hardware processor; and
a non-transitory computer-readable storage medium having stored thereon program instructions, the program instructions executable by the at least one hardware processor to:
(i) operate said light beam source to illuminate a tissue sample with a first light beam having a wavelength selected from the range of 390-430 nanometers (nm), wherein said tissue sample is applied with a contrast agent, and
(ii) operate said imaging device to capture one or more images of the tissue sample;
wherein tissue abnormality is detectable based on at least one of color changes in the tissue sample and blood vessel features in the tissue sample appearing in said one or more images.

32. (canceled)

33. (canceled)

34. The system of claim 31, wherein said imaging device is configured for capturing said one or more images along a direct optical path from said tissue sample.

35. The system of claim 31, further comprising alight guide configured for directing at least one of reflectance and fluorescence from the tissue sample to the imaging device.

36. The system of claim 31, wherein said light beam source is configured for illuminating said tissue sample directly.

37. (canceled)

38. The system of claim 31, wherein said instructions comprise operating said imaging device to capture at least one of said one or more images before said tissue sample is applied with said contrast agent, and to capture at least one of said one or more images after said tissue sample is applied with said contrast agent, wherein said detecting is based at least in part on a comparison between said images captured before and after the step of applying said contrast agent.

39. The system of claim 31, wherein said instructions comprise operating said imaging device to capture at least some of said one or more images at a specified time period after said tissue sample is applied with said contrast agent, and wherein said specified time period is between 1 and 600 seconds.

40. The system of claim 31, wherein at least some of said one or more images are RGB images, wherein said instructions comprise changing one or more amplification ratios between RGB channels of said RGB images, and wherein said detecting is further based on detecting fluorescence emitted by said tissue sample appearing in at least some of said one or more images.

41. The system of claim 40, wherein said fluorescence is in at least one wavelength selected from the ranges 490-580 nm and 600-750 nm.

42. The system of claim 31, further comprising a second light source configured for illuminating said tissue sample with a second light beam, wherein said second light beam has a different wavelength to than said first light beam.

43. The system of claim 42, wherein said instructions further comprise operating said first and second light sources for illuminating said tissue sample simultaneously with said first light beam and said second light beam.

44. The system of claim 42, wherein said instructions further comprise operating said first and second light sources for illuminating said tissue sample sequentially with said first light beam and said second light beam, wherein at least some of said one or more images are being captured during a period for which said first light beam is illuminating the tissue sample, and at least some of said one or more images are being captured during a period for which said second light beam is illuminating the tissue sample.

45. The system of claim 42, further comprising two or more imaging devices,
wherein each of said imaging devices is configured for acquiring image data in different one or more spectral bands.

46. The system of claim 45, further comprising at least one of:
confocal imaging means;
a dichroic mirror having a cutoff wavelength selected from the group consisting of 430 nm, 580-660 nm, and 800 nm; and
a beam splitter.

47. (canceled)

48. (canceled)

49. The system of claim 42, wherein said second light beam has a wavelength selected from the range 495-570 nm, and wherein said detecting is further based on detecting blood vessel features below a superficial layer of said tissue sample appearing in at least some of said one or more images.

50. The system of claim 42, further comprising a third light source configured for illuminating said tissue sample with a third light beam, wherein each of said second and third light beams has a wavelength selected from the range 585-720 nm, and wherein said detecting is further based on determining a value of oxygen saturation of the blood in said tissue sample.

51. The system of claim 42, wherein said second light beam has a wavelength selected from the range 900-3000 nm, and wherein said detecting is further based on determining a value of fluid accumulation in said tissue sample.

52. The system of claim 42, wherein said second light beam has a wavelength selected from the range 100-390 nm, and wherein said detecting is further based on measuring fluorescence emitted by one or more excited fluorophores in said tissue sample.

53. The system of claim 42, further comprising a projection system configured for illuminating said tissue sample with spatially-structured light, and wherein said detecting is further based, at least in part, on depth-resolved measuring of one or more of tissue blood concentration, tissue blood oxygenation, tissue water fraction, tissue perfusion, collagen, lipids, and exogenous agents.

54. The system of claim 53, wherein said spatially-structured light is configured for performing spatial frequency domain imaging (SFDI).

55. The system of claim 53, wherein said depth-resolved measuring comprises adjusting at least one of light frequency, wavelength selection, and amplitude modulation of said spatially-structured light.

56-60. (canceled)
